(19) United States
(12) Patent Application Publication
(10) Pub. No.: US 2026/0345454 A1
(43) Pub. Date: Nov. 5, 2026

(54) DETERGENT AND EXFOLIATING COSMETIC HAIR COMPOSITION COMPRISING SALICYLIC ACID AND PARTICLES IN SUSPENSION

(71) Applicant: L'Oreal, Paris (FR)

(72) Inventors: Nathalie Savitch, Saint Ouen (FR); Léa Chabas, Saint Ouen (FR); Mathieu Chabrillangeas, Chevilly (FR)

(73) Assignee: L'Oreal, Paris (FR)

(21) Appl. No.: 19/119,654

(22) PCT Filed: Oct. 4, 2023
(86) PCT No.: PCT/EP2023/077441

§ 371 Date: Apr. 9, 2025

(30) Foreign Application Priority Data
Oct. 10, 2022 (FR) FR2210346

(51) Int. Cl. A61K 8/368; A61K 8/365; A61K 8/44; A61K 8/46; A61K 8/81; A61K 8/9789; A61Q 5/00; A61Q 5/02; A61Q 5/06; A61Q 5/12

(52) U.S. Cl. CPC A61K 8/368; A61K 8/365; A61K 8/442; A61K 8/463; A61K 8/8147; A61K 8/9789; A61Q 5/006; A61Q 5/02; A61Q 5/06; A61Q 5/12; A61K 2800/34; A61K 2800/41; A61K 2800/5424

(57) ABSTRACT

The present invention relates to a cosmetic composition comprising (i) salicylic acid and/or at least one salicylic acid salt, in a content of at least 1.5% by weight, (ii) solid particles with a number-average size of greater than or equal to 300 μm, (iii) at least one anionic surfactant, (iv) at least one amphoteric or zwitterionic surfactant, and (v) at least one crosslinked anionic copolymer of at least one monomer A chosen from vinyl carboxylic acids and of at least one monomer B containing at least one α,β-ethylenic unsaturation. The invention also relates to a process for treating keratin materials, comprising at least one step of applying such a composition to said materials.

The present invention relates to a cosmetic hair composition with detergent and exfoliating action, which comprises salicylic acid or a salt thereof, at least one anionic surfactant, at least one amphoteric or zwitterionic surfactant, solid particles and at least one particular anionic acrylic copolymer.

The invention also relates to a process for treating keratin materials, comprising at least one step of applying a composition according to the invention to said materials.

It is common practice to use detergent compositions (such as shampoos), based essentially on surfactants, for washing and/or cleansing keratin materials such as the hair and the scalp. These compositions are applied to wet hair and the foam generated by massaging or rubbing with the hands makes it possible, after rinsing with water, to remove the diverse types of soiling initially present on the hair and the scalp.

However, these compositions do not always allow soiling to be completely removed and/or a clean visual effect to be obtained. In particular, in the case of users with a scalp that tends to be greasy and/or gives rise to desquamation, traditional detergent compositions do not always allow organic matter (sebum, dandruff, etc.) formed on the surface of the scalp and/or clinging to the hair to be completely removed. There is thus a need to develop cosmetic compositions for treating and notably washing keratin materials, which allow organic materials, notably dandruff, to be removed when present, and to prevent them from appearing.

Moreover, consumers are in search of cosmetic compositions which contain higher proportions of natural and biodegradable ingredients, advantageously silicone-free compositions, which have improved working qualities and good cosmetic properties, and which are capable of washing keratin materials without making them feel heavy, so as to provide good conditioning properties to the keratin fibres.

In parallel, users of cosmetic products are often in search of hair hygiene products that are more aesthetic or even more original.

There is thus a real need to develop aesthetic detergent cosmetic compositions which are stable over time, based on natural and/or biodegradable ingredients, which preferably do not comprise silicone, and which make it possible to obtain excellent washing properties. These compositions must also have good working properties, in particular in terms of the start of lathering, the quality and amount of foam generated, and viscosity. It is also advantageous for these compositions to confer good conditioning properties on keratin fibres, notably in terms of feel, smoothness, suppleness and disentangling.

These objectives are achieved by the present invention, one subject of which is a composition, preferably a cosmetic composition, comprising:
(i) salicylic acid and/or at least one salicylic acid salt, in a total content of at least 1.5% by weight relative to the total weight of the composition,
(ii) solid particles with a number-average size of greater than or equal to 300 μm, in a total content of at least 0.2% by weight relative to the total weight of the composition,
(iii) at least one anionic surfactant,
(iv) at least one amphoteric or zwitterionic surfactant, and
(v) at least one crosslinked anionic copolymer of at least one monomer A chosen from vinyl carboxylic acids and of at least one monomer B containing at least one α,β-ethylenic unsaturation chosen from monomers of the following formulae:

CH₂═CXY b1)
where X denotes hydrogen and Y denotes a group chosen from —COOR, —C₆H₄R′, —CN, —CONH₂, —NHCOCH₃, —CONHC(CH₃)₃, and —CON(CH₃)₂, or
X denotes CH₃ and Y denotes a group chosen from —COOR, —C₆H₄R′, —CN or —CH═CH₂,
R denoting a C₁-C₈ alkyl group or a C₂-C₈ hydroxyalkyl group, preferably a C₁-C₄ alkyl group or a C₂-C₄ hydroxyalkyl group,
R′ denoting hydrogen, a C₁-C₈ alkyl group or a C₂-C₈ hydroxyalkyl group;

CH₂═CH(OCOR′) b2)
with R¹ denoting a C₁-C₈ and preferably C₁-C₄ alkyl group;

CH₂═CH₂ or CH₂═CHCH₃. b3)

The composition according to the invention has excellent washing power on keratin materials. It allows soiling to be effectively removed and a clean visual effect to be obtained. In addition, the composition according to the invention has an exfoliating effect on the scalp, which is immediately perceptible on application, allowing organic matter, notably sebum and tissue fragments liable to form dandruff, to be effectively removed. It also allows dandruff clinging to keratin fibres to be eliminated.

The composition according to the invention also has a keratolytic action, which in combination with the exfoliating effect described above allows the scalp to be effectively prevented from becoming greasy again and dandruff from forming. The keratin materials remain clean for longer and the average interval between two shampoo washes may be increased.

It has also been noted that the hair thus treated is particularly light, has a soft and smooth feel, and is supple, easy to disentangle and manageable.

It has also been found that the composition according to the invention has good working qualities, notably a good start of lathering, and good foam quality and quantity.

The composition according to the invention has a viscosity that is suitable for use as a shampoo.

Moreover, it has also been found that the composition according to the invention is stable over time, at atmospheric pressure and at 25 °C.

More particularly, the solid particles remain in homogeneous and stable suspension, and no phase separation of the particles within the composition has been observed. The visually stable and homogeneous suspension of these particles substantially improves the aesthetic appearance and the originality of the composition according to the invention.

For the purposes of the invention, the term “stable and homogeneous suspension” means that said particles are homogeneously dispersed in the composition, that is to say homogeneously distributed within the composition, and that this homogeneous dispersion persists over time, that is to say that the particles do not float at the surface, do not sink, do not sediment out and/or do not form agglomerates in the composition over time, in particular after at least one month of storage.

Advantageously, the composition comprising the suspended particles is clear.

A subject of the invention is also a treatment process, notably a cosmetic treatment process, for keratin materials, in particular human keratin materials such as the hair and the scalp, comprising at least one step of applying a composition according to the invention to said keratin materials.

Other subjects, characteristics, aspects and advantages of the invention will emerge even more clearly on reading the description and the example that follows.

In the present description, and unless otherwise indicated:
the term “at least one” is equivalent to the expression “one or more” and can be replaced therewith;
the term “between . . . and . . . ” is equivalent to the term “ranging from . . . to . . . ” and can be replaced therewith, and implies that the limits are included;
the term “greater than” and, respectively, the term “less than” refer to an open range which is strictly greater, or, respectively, strictly less, and thus that the limits are not included;
the term “keratin materials” more particularly denotes human keratin materials, and more preferentially the hair and the scalp;
the term “fatty acid” means an organic acid comprising in its structure a linear or branched, saturated or unsaturated hydrocarbon-based chain comprising from 6 to 40 carbon atoms, preferably from 8 to 30 carbon atoms, more preferentially from 10 to 22 carbon atoms;
the term “fatty alcohol” means an alcohol comprising in its structure a linear or branched, saturated or unsaturated hydrocarbon-based chain comprising from 6 to 40 carbon atoms, preferably from 8 to 30 carbon atoms, more preferentially from 10 to 22 carbon atoms.

Preferably, the composition according to the invention is silicone-free.

The term “silicone free” means that the silicone(s) optionally present in the composition are included in a total content of less than or equal to 0.1% by weight, preferably less than or equal to 0.05% by weight, more preferentially less than or equal to 0.01% by weight, relative to the total weight of the composition, and better still that the composition is free of silicone (0% by weight).

The term “silicone” means any organosilicon polymer or oligomer of linear or cyclic, and branched or crosslinked structure, of variable molecular weight, obtained, for example, by polymerization and/or polycondensation of suitably functionalized silanes and consisting essentially of a repetition of main units in which the silicon atoms are connected to each other via oxygen atoms (siloxane bond —Si—O—Si—), optionally substituted hydrocarbon-based radicals being connected directly to said silicon atoms via a carbon atom; and more particularly dialkylsiloxane polymers, amino silicones and dimethiconols.

### Salicylic Acid and Salts Thereof

The composition according to the invention comprises one or more compounds chosen from salicylic acid and salicylic acid salts.

The term “salicylic acid salt” denotes any compound in which salicylic acid is in anionic form, combined with any mineral or organic cation. Use is preferably made of salts derived from a mineral or organic base.

Examples of mineral bases that may notably be mentioned include alkali metal or alkaline-earth metal hydroxides, for instance sodium hydroxide or potassium hydroxide, or aqueous ammonia.

Organic bases that may notably be mentioned include amines and alkanolamines.

Preferably, the composition comprises one or more compounds chosen from salicylic acid and the sodium or potassium salts of salicylic acid. Even more preferentially, the composition contains salicylic acid.

Preferably, the composition contains salicylic acid and/or at least one salicylic acid salt in a total content ranging from 1.5% to 8% by weight, preferably ranging from 1.8% to 6% by weight and better still from 2% to 5% by weight, relative to the total weight of the composition.

Preferably, the composition contains salicylic acid in a content of at least 1.5% by weight, better still ranging from 1.5% to 8% by weight, preferably from 1.8% to 6% by weight, and better still from 2% to 5% by weight relative to the total weight of the composition.

### Particles

The composition according to the present invention also comprises one or more solid particles.

The term “solid particles” means small solid objects which may be of variable shapes and sizes. They may be in regular or irregular form. They may in particular be in spherical form (such as granules, granulates or beads) or in square, rectangular or elongated form such as sticks.

The composition according to the invention comprises a total content of at least 0.2% by weight, relative to the total weight of the composition, of solid particles with a number-average size greater than or equal to 300 μm, preferably in the range from 300 to 1500 μm, preferably from 350 to 1000 μm, better still from 400 to 800 μm.

The number-average particle size is expressed relative to the largest number-average dimension of said particles.

These particle sizes may be measured with a laser granulometer (for instance using a Brookhaven BI90 or Malvern device).

These particle sizes may also be measured using granulometric analysis sieves.

The solid particles that may be used in the composition according to the invention may be chosen from mineral particles and organic particles.

The solid particles are preferably chosen from organic particles and more preferentially from organic particles of plant origin, notably fragments of one or more plants.

For the purposes of the present invention, the term “fragment” means a piece or a part of a plant, obtained, for example, by tearing or milling the plant, or by cutting up said plant.

For the purposes of the present invention, the term “fragments of one or more plants” means that the composition comprises either several fragments of the same species of plant, or that the composition comprises several fragments of several different species of plants.

Preferably, the composition comprises plant fragments in the form of plant material powder, obtained, for example, by grinding these materials. The plant materials may thus be, for example, plant kernels, pips, shells or seeds.

Preferably, the composition according to the invention comprises at least one powder of plant origin, notably at least one plant material powder, more preferentially chosen from kernel powders, for instance apricot kernel powder, plum stone powder and olive stone powder; nut shell powders, for instance argan nut shell powder (INCI name Argania spinosa shell powder), walnut shell powder; seed powders, for instance coffee bean powder, cocoa bean powder; seed powders, for instance kiwi seed powder; and also mixtures of such powders.

Preferably, the composition according to the invention comprises at least two types of powders of different plant materials, for instance a kernel powder and a nut shell powder; better still an apricot kernel powder and an argan nut shell powder. Advantageously, these powders have different particle sizes.

Preferably the composition according to the invention comprises one or more solid particles with a number-average size ranging from 300 to 400 μm and one or more solid particles with a number-average size greater than 400 μm, more preferentially ranging from 410 to 800 μm.

Preferably, the composition according to the invention comprises one or more powders of plant materials with a number-average size ranging from 300 to 400 μm and one or more powders of plant materials with a number-average size greater than 400 μm, more preferentially ranging from 410 to 800 μm.

Even better still, the composition according to the invention comprises one or more kernel powders, notably apricot kernel powder, with a number-average size ranging from 300 to 400 μm and one or more nut shell powders, notably argan nut shell powder, with a number-average size greater than 400 μm, more preferentially ranging from 410 to 800 μm.

Preferably, the composition comprises said solid particles in a total content ranging from 0.2% to 10% by weight, more preferentially from 0.5% to 5% by weight, better still from 0.7% to 2% by weight, and even better still from 0.8% to 1.5% by weight, relative to the total weight of the composition.

Preferably, the composition comprises said powders of plant materials in a total content ranging from 0.2% to 10% by weight, more preferentially from 0.5% to 5% by weight, better still from 0.7% to 2% by weight, and even better still from 0.8% to 1.5% by weight, relative to the total weight of the composition.

### Anionic Surfactants

The composition according to the present invention also comprises one or more anionic surfactants.

The term “anionic surfactant” means a preferably non-silicone surfactant including, as ionic or ionizable groups, only anionic groups.

In the present description, a species is termed as being “anionic” when it bears at least one permanent negative charge or when it can be ionized to a negatively charged species, under the conditions of use of the composition of the invention (for example the medium or the pH) and not comprising any cationic charge.

The anionic surfactants may be chosen from sulfate, sulfonate and carboxylic (or carboxylate) surfactants. Needless to say, a mixture of these surfactants may be used.

It is understood in the present description that:
the carboxylate anionic surfactants comprise at least one carboxylic or carboxylate function (—COOH or —COO—) and do not comprise any sulfate and/or sulfonate functions;
the sulfonate anionic surfactants comprise at least one sulfonate function (—SO₃H or —SO₃⁻) and may optionally also comprise one or more carboxylate functions, but do not comprise any sulfate functions; and
the sulfate anionic surfactants comprise at least one sulfate function but do not comprise any carboxylate or sulfonate functions.

The carboxylate anionic surfactants that may be used thus include at least one carboxylic or carboxylate function (—COOH or —COO—).

The carboxylate anionic surfactants may be chosen from the following compounds: acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and mixtures thereof;
the alkyl and/or acyl groups of these compounds including from 6 to 30 carbon atoms, notably from 12 to 28, even better still from 14 to 24 or even from 16 to 22 carbon atoms; the aryl group preferably denoting a phenyl or benzyl group;
it being possible for these compounds to be polyoxyalkylenated, particularly polyoxyethylenated, and then preferably including from 1 to 50 ethylene oxide units and better still from 2 to 10 ethylene oxide units.

Use may also be made of C₆-C₂₄ alkyl monoesters of polyglycoside-polycarboxylic acids such as C₆-C₂₄ alkyl polyglycoside-citrates, C₆-C₂₄ alkyl polyglycoside-tartrates and C₆-C₂₄ alkyl polyglycoside-sulfosuccinates, and salts thereof.

Preferentially, the carboxylate anionic surfactants are chosen, alone or as a mixture, from:
acyl glutamates, notably C₆-C₂₄ or even C₁₂-C₂₀ acyl glutamates, such as stearoyl glutamates, and in particular disodium stearoyl glutamate;
acyl sarcosinates, notably C₆-C₂₄ or even C₁₂-C₂₀ acyl sarcosinates, such as palmitoyl sarcosinates, and in particular sodium palmitoyl sarcosinate;
acyl lactylates, notably C₁₂-C₂₈ or even C₁₄-C₂₄ acyl lactylates, such as behenoyl lactylates, and in particular sodium behenoyl lactylate;
C₆-C₂₄ and notably C₁₂-C₂₀ acylglycinates;
(C₆-C₂₄)alkyl ether carboxylates, and notably (C₁₂-C₂₀)alkyl ether carboxylates;
polyoxyalkylenated (C₆-C₂₄)alkyl(amido) ether carboxylic acids, in particular those including from 2 to 50 ethylene oxide groups;
in particular in the form of alkali metal or alkaline-earth metal, ammonium or amino alcohol salts.

Surfactants of the sarcosinate type may notably be chosen from alkyl(C₆-C₃₀)sarcosinates of formula (I) below:

R—C(O)—N(CH₃)—CH₂—C(O)—OX (I)
with
X denoting a hydrogen atom, an ammonium ion, an ion derived from an alkali metal or an alkaline-earth metal or an ion derived from an organic amine, preferably a hydrogen atom, and
R denoting a linear or branched alkyl group of 5 to 29 carbon atoms.

Preferably, R denotes a linear or branched alkyl group of 8 to 24 carbon atoms, preferably of 12 to 20 carbon atoms.

Among the (C₆-C₃₀)acyl sarcosinates of formula (I) that may be used in the present composition, mention may be made of palmitoyl sarcosinates, stearoyl sarcosinates, myristoyl sarcosinates, lauroyl sarcosinates and cocoyl sarcosinates, in acid form or in salified form.

The anionic surfactant(s) of sarcosinate type are advantageously chosen from sodium lauroyl sarcosinate, stearoylsarcosine, myristoylsarcosine, and mixtures thereof, preferably from stearoylsarcosine, myristoylsarcosine, and mixtures thereof.

Among the above carboxylic surfactants, mention may also be made of polyoxyalkylenated alkyl(amido) ether carboxylic acids and salts thereof, in particular those including from 2 to 50 alkylene oxide and in particular ethylene oxide groups, such as the compounds sold by the company Kao under the Akypo names.

The polyoxyalkylenated alkyl(amido) ether carboxylic acids that may be used are preferably chosen from those of formula (II):

R1-(OC₂H₄)ₙ—OCH₂COOA (II)
in which:
R1 represents a linear or branched C₆-C₂₄ alkyl or alkenyl radical, a (C₈-C₉)alkylphenyl radical, a radical R₂CONH—CH₂—CH₂— with R2 denoting a linear or branched C₉-C₂₁ alkyl or alkenyl radical;
preferably, R1 is a C₈-C₂₀ and preferably C₈-C₁₈ alkyl radical, and aryl preferably denotes phenyl,
n is an integer or decimal number (mean value) ranging from 2 to 24 and preferably from 2 to 10,
A denotes H, ammonium, Na, K, Li, Mg or a monoethanolamine or triethanolamine residue.

Use may also be made of mixtures of compounds of formula (II), in particular mixtures of compounds bearing different groups R1.

The polyoxyalkylenated alkyl(amido) ether carboxylic acids that are preferred are those of formula (II) in which:
R1 denotes a C₁₂-C₁₄ alkyl, cocoyl, oleyl, nonylphenyl or octylphenyl radical,
A denotes a hydrogen or sodium atom, and
n ranges from 2 to 20, preferably from 2 to 10.

Even more preferentially, R1 denotes a C₁₂ alkyl radical, A denotes a hydrogen or sodium atom and n ranges from 2 to 10.

The sulfonate anionic surfactants that may be used include at least one sulfonate function (—SO₃H or —SO₃⁻).

The sulfonate anionic surfactants may be chosen from the following compounds: alkylsulfonates, alkylamidesulfonates, alkylarylsulfonates, α-olefin sulfonates, paraffin sulfonates, alkyl sulfosuccinates, alkyl ether sulfosuccinates, alkylamidesulfosuccinates, alkylsulfoacetates, N-acyltaurates, acylisethionates, alkylsulfolaurates; and also the salts of these compounds;
the alkyl groups of these compounds including from 6 to 30 carbon atoms, notably from 12 to 28, even better still from 14 to 24 or even from 16 to 22 carbon atoms; the aryl group preferably denoting a phenyl or benzyl group;
it being possible for these compounds to be polyoxyalkylenated, particularly polyoxyethylenated, and then preferably including from 1 to 50 ethylene oxide units and better still from 2 to 10 ethylene oxide units.

Preferentially, the sulfonate anionic surfactants are chosen, alone or as a mixture, from:
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
C₆-C₂₄ and notably C₁₂-C₂₀ N-acyltaurates;
(C₆-C₂₄)acyl isethionates, preferably (C₁₂-C₁₈)acyl isethionates;
in particular in the form of alkali metal or alkaline-earth metal, ammonium or amino alcohol salts.

Preferably, the anionic surfactant(s) of sulfonate type are chosen from C₆-C₂₄ and notably C₁₂-C₂₀ N-acyltaurates, and in particular N-acyl N-methyltaurates, C₆-C₂₄ and notably C₁₂-Cis acylisethionates, and also salts thereof and mixtures thereof.

More preferentially, the anionic surfactant(s) of sulfonate type are chosen from C₆-C₂₄ and notably C₁₂-Cis acylisethionates, and also salts thereof and mixtures thereof.

The sulfate anionic surfactants that may be used include at least one sulfate function (—OSO₃H or —OSO₃⁻).

The sulfate anionic surfactants may be chosen from the following compounds: alkyl sulfates, alkyl ether sulfates, alkylamido ether sulfates, alkylaryl polyether sulfates, monoglyceride sulfates; and the salts of these compounds;
the alkyl groups of these compounds including from 6 to 30 carbon atoms, notably from 8 to 28, even better still from 10 to 24 or even from 12 to 22 carbon atoms; the aryl group preferably denoting a phenyl or benzyl group;
these compounds possibly being (poly)oxyalkylenated, notably (poly)oxyethylenated, and then preferably including from 1 to 50 ethylene oxide units and better still from 1 to 10 ethylene oxide units.

Preferentially, the sulfate anionic surfactants are chosen, alone or as a mixture, from:
alkyl sulfates, notably C₁₀-C₂₄ or even C₁₂-C₂₂ alkyl sulfates;
alkyl ether sulfates, notably C₁₀-C₂₄ or even C₁₂-C₂₂ alkyl ether sulfates, preferably comprising from 1 to 20 ethylene oxide units;
in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-propane-1,3-diol salts and tris(hydroxymethyl)aminomethane salts.

Alkali metal or alkaline-earth metal salts and in particular sodium or magnesium salts are preferably used.

Advantageously, the anionic surfactant(s) are chosen from sulfate anionic surfactants, carboxylate anionic surfactants and mixtures of these two types of surfactants.

Preferably, the composition according to the invention comprises at least one carboxylate anionic surfactant, more preferentially chosen from: acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and mixtures thereof;
the alkyl and/or acyl groups of these compounds including from 6 to 30 carbon atoms, notably from 12 to 28, even better still from 14 to 24 or even from 16 to 22 carbon atoms; the aryl group preferably denoting a phenyl or benzyl group; it being possible for these compounds to be polyoxyalkylenated, particularly polyoxyethylenated, and then preferably including from 1 to 50 ethylene oxide units and better still from 2 to 10 ethylene oxide units.

In a particularly preferred manner, the composition comprises at least one carboxylate anionic surfactant chosen from (C₆-C₂₄)alkyl ether carboxylic acids, and notably (C₁₂-C₂₀)alkyl ether carboxylic acids.

Advantageously, the composition according to the invention comprises at least one sulfate anionic surfactant, preferably chosen from alkyl sulfates, alkyl ether sulfates, alkyl amido ether sulfates, alkyl aryl polyether sulfates, monoglyceride sulfates, the salts of these compounds, and mixtures thereof;
the alkyl groups of these compounds preferably comprising from 6 to 30 carbon atoms, notably from 8 to 28, even better still from 10 to 24 or even from 12 to 22 carbon atoms; the aryl group preferably denoting a phenyl or benzyl group;
these compounds possibly being (poly)oxyalkylenated, notably (poly)oxyethylenated, and then preferably including from 1 to 50 ethylene oxide units and better still from 1 to 10 ethylene oxide units.

More preferentially, the anionic surfactant(s) are chosen from:
C₆-C₃₀, better still C₅-C₂₄, even better still C₁₀-C₂₄ or even C₁₂-C₂₂ alkyl sulfates,
C₆-C₂₄, better still C₁₀-C₂₄ or even C₁₂-C₂₂ alkyl ether sulfates, preferably comprising from 1 to 20 ethylene oxide units,
the salts of these compounds and mixtures thereof;
in particular in the form of alkali metal or alkaline-earth metal, ammonium or amino alcohol salts.

Even more preferentially, the anionic surfactant(s) are chosen from C₆-C₃₀, better still C₅-C₂₄, even better still C₁₀-C₂₄ or even C₁₂-C₂₂ alkyl sulfates.

Particularly preferably, the composition according to the invention comprises at least one carboxylate anionic surfactant and at least one sulfate anionic surfactant as described above.

Preferably, the total content of anionic surfactant(s) ranges from 1% to 25% by weight, more preferentially from 2% to 20% by weight, even more preferentially from 5% to 18% by weight and better still from 10% to 18% by weight relative to the total weight of the composition.

Preferably, the total content of carboxylate anionic surfactant(s) present in the composition ranges from 0.5% to 10% by weight and more preferentially from 1% to 5% by weight, relative to the total weight of the composition.

More preferentially, the total content of carboxylate anionic surfactant(s) chosen from C₆-C₂₄ alkyl ether carboxylic acids, and notably C₁₂-C₂₀ alkyl ether carboxylic acids, ranges from 0.5% to 10% by weight, more preferentially from 1% to 5% by weight, relative to the total weight of the composition.

Preferably, the total content of sulfate anionic surfactant(s) present in the composition ranges from 1% to 20% by weight, more preferentially from 5% to 18% by weight, more preferably from 10% to 15% by weight, relative to the total weight of the composition.

Preferably, the total content of sulfate anionic surfactant(s) chosen from C₆-C₃₀ alkyl sulfates and C₆-C₂₄ alkyl ether sulfates ranges from 1% to 20% by weight, more preferentially from 5% to 18% by weight, better still from 10% to 15% by weight, relative to the total weight of the composition.

### Amphoteric Surfactants

The composition according to the present invention comprises one or more amphoteric or zwitterionic surfactants.

In particular, the amphoteric or zwitterionic surfactant(s), which are preferably non-silicone, used in the composition according to the present invention may notably be derivatives of optionally quaternized aliphatic secondary or tertiary amines, in which derivatives the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₁-C₆)alkylbetaines and (C₈-C₂₀)alkylamido(C₁-C₆)alkylsulfobetaines, and mixtures thereof.

Among the optionally quaternized derivatives of secondary or tertiary aliphatic amines that may be used, as defined above, mention may also be made of the compounds having the respective structures (III) and (IV) below:

Rₐ—CONHCH₂CH₂—N⁺(R_{b})(R_{c})—CH₂COO⁻,M⁺,X⁻ (III)
in which formula (III):
Rₐ represents a C₁₀ to C₃₀ alkyl or alkenyl group derived from an acid RₐCOOH which is preferably present in hydrolysed coconut kernel oil; preferably, Rₐ represents a heptyl, nonyl or undecyl group;
R_{b} represents a β-hydroxyethyl group;
R_{c} represents a carboxymethyl group;
M⁺ represents a cationic counterion derived from an alkali metal or alkaline-earth metal, such as sodium, an ammonium ion or an ion derived from an organic amine; and
X⁻ represents an organic or inorganic anionic counterion, such as that chosen from halides, acetates, phosphates, nitrates, (C₁-C₄)alkyl sulfates, (C₁-C₄)alkyl- or (C₁-C₄)alkylaryl-sulfonates, in particular methyl sulfate and ethyl sulfate; or alternatively M⁺ and X⁻ are absent;
Rₐ′—CONHCH₂CH₂—N(B)(B′) (IV) in which formula (IV):
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
Rₐ′ represents a C₁₀ to C₃₀ alkyl or alkenyl group of an acid Rₐ′—COOH preferably present in hydrolysed linseed oil or coconut kernel oil; preferably, Rₐ′ is an alkyl group, in particular a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (V):

Rₐ″—NHCH(Y″)—(CH₂)ₙCONH(CH₂)ₙ′—N(R_{d})(Rₑ) (V)
in which formula (V):
Y″ represents the group —COOH, —COOZ″ or —CH₂—CH(OH)SO₃H or the group CH₂CH(OH)SO₃—Z″;
R_{d} and Rₑ, independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
Rₐ″ represents a C₁₀ to C₃₀ alkyl or alkenyl group of an acid Rₐ″—COOH preferably present in hydrolysed linseed oil or coconut kernel oil; and
n and n′ denote, independently of each other, an integer ranging from 1 to 3.

Among the compounds of formula (V), mention may be made of the compound classified in the CTFA dictionary under the name sodium diethylaminopropyl cocoaspartamide and sold by the company Chimex under the name Chimexane HB.

These compounds may be used alone or as mixtures.

Among the amphoteric or zwitterionic surfactants mentioned above, use is advantageously made of (C₈-C₂₀)alkylbetaines, such as cocoyl betaine, (C₈-C₂₀)alkylamido(C₃-C₈)alkylbetaines, such as cocamidopropylbetaine, (C₈-C₂₀)alkylamphoacetates, (C₅-C₂₀)alkylamphodiacetates and mixtures thereof.

More preferentially, the amphoteric or zwitterionic surfactants are chosen from (C₈-C₂₀)alkylbetaines, (C₈-C₂₀)alkylamido(C₃-C₈)alkylbetaines, and mixtures thereof, even more preferentially from cocoyl betaine, cocamidopropylbetaine, and mixtures thereof.

Better still, the amphoteric or zwitterionic surfactants are chosen from (C₈-C₂₀)alkylbetaines and most particularly cocoyl betaine.

Preferably, the total content of amphoteric or zwitterionic surfactant(s) present in the composition ranges from 0.1% to 15% by weight, more preferentially from 0.5% to 10% by weight, even more preferentially from 1% to 5% by weight relative to the total weight of the composition.

Preferably, the total content of (C₈-C₂₀)alkylbetaine(s) and (C₈-C₂₀)alkylamido(C₃-C₈)alkylbetaine(s) present in the composition ranges from 0.1% to 15% by weight, more preferentially from 0.5% to 10% by weight, even more preferentially from 1% to 5% by weight relative to the total weight of the composition.

More preferentially, the total content of (C₅-C₂₀)alkylbetaine(s) present in the composition ranges from 0.1% to 15% by weight, more preferentially from 0.5% to 10% by weight and even more preferentially from 1% to 5% by weight, relative to the total weight of the composition.

Advantageously, the total content of anionic surfactant(s) and amphoteric or zwitterionic surfactant(s) present in the composition is greater than or equal to 5% by weight, more preferentially greater than or equal to 10% by weight and better still greater than or equal to 15% by weight relative to the total weight of the composition.

Preferably, the total content of anionic surfactant(s) and amphoteric or zwitterionic surfactant(s) present in the composition ranges from 5% to 40% by weight, more preferentially from 10% to 25% by weight and even more preferentially from 15% to 20% by weight relative to the total weight of the composition.

### The Crosslinked Anionic Copolymer

The composition according to the invention comprises one or more crosslinked anionic copolymers. The copolymer(s) used in the composition according to the invention are derived from one or more monomers A chosen from vinyl carboxylic acids, one or more monomers B containing at least one α,β-ethylenic unsaturation and one or more crosslinking agents.

The monomers A are preferably chosen from acrylic acid, methacrylic acid, itaconic acid, fumaric acid, crotonic acid, maleic acid and mixtures thereof, more preferentially from acrylic acid and methacrylic acid, and better still methacrylic acid.

The monomers B containing at least one α,β-ethylenic unsaturation are chosen from the monomers having the formulae:

CH₂═CXY b1)
where X denotes hydrogen and Y denotes a group chosen from —COOR, —C₆H₄R′, —CN, —CONH₂, —NHCOCH₃, —CONHC(CH₃)₃, and —CON(CH₃)₂, or
X denotes CH₃ and Y denotes a group chosen from —COOR, —C₆H₄R′, —CN or —CH═CH₂,
R denoting a C₁-C₈ alkyl group or a C₂-C₈ hydroxyalkyl group, preferably a C₁-C₄ alkyl group or a C₂-C₄ hydroxyalkyl group,
R′ denoting hydrogen, a C₁-C₈ alkyl group or a C₂-C₈ hydroxyalkyl group;

CH₂═CH(OCOR′) b2)
with R¹ denoting a C₁-C₈ and preferably C₁-C₄ alkyl group;

CH₂═CH₂ or CH₂═CHCH₃. b3)

Preferably, R denotes a C₁-C₄ alkyl group or a C₂-C₄ hydroxyalkyl group.

The monomer B containing at least one α,β-ethylenic unsaturation is preferably a monomer of formula b1).

Among these monomers B, mention may be made of methyl acrylate, ethyl acrylate, n-butyl acrylate, 2-ethylhexyl acrylate, 2-hydroxyethyl acrylate, styrene, vinyl acetates, acrylamide, N,N-dimethylacrylamide, tert-butylacrylamide, methyl methacrylate, ethyl methacrylate, n-butyl methacrylate, 2-ethylhexyl methacrylate and 2-hydroxyethyl methacrylate.

The monomer B containing at least one α,β-ethylenic unsaturation is preferably a monomer of formula b1) in which X denotes hydrogen and Y denotes —COOR with R denoting a C₁-C₈, better still C₁-C₄, alkyl group or a C₂-C₈, better still C₂-C₄, hydroxyalkyl group.

More preferentially, the monomer B is chosen from methyl acrylate, ethyl acrylate and butyl acrylate and more particularly ethyl acrylate.

The monomer A is preferably present in amounts ranging from 20% to 80% by weight, more particularly from 25% to 70% by weight and even more particularly from 35% to 60% by weight relative to the total weight of the copolymer.

The monomer B is preferably present in amounts ranging from 15% to 80% by weight, more particularly from 25% to 75% by weight and even more particularly from 40% to 65% by weight relative to the total weight of the copolymer.

This copolymer is partially or totally crosslinked with at least one conventional crosslinking agent. The crosslinking agents are notably polyunsaturated compounds, in particular ethylenically polyunsaturated compounds. These compounds are notably polyalkenyl ethers of sucrose or of polyols, diallyl phthalates, divinylbenzene, allyl (meth)acrylate, ethylene glycol di(meth)acrylate, methylenebisacrylamide, trimethylolpropane tri(meth)acrylate, diallyl itaconate, diallyl fumarate, diallyl maleate, zinc (meth)acrylate, and castor oil or polyol derivatives manufactured from unsaturated carboxylic acids.

The crosslinking agent may also be chosen from unsaturated monomeric compounds including a reactive group that is capable of reacting with an unsaturation to form a crosslinked copolymer.

The content of crosslinking agent generally ranges from 0.01% to 5% by weight, preferably from 0.03% to 3% by weight and even more particularly from 0.05% to 1% by weight relative to the total weight of the copolymer.

Preferably, the crosslinked anionic copolymer is chosen from crosslinked copolymers of methacrylic acid and of a C₁-C₄ alkyl acrylate, crosslinked copolymers of acrylic acid and of a C₁-C₄ alkyl acrylate, and more preferentially crosslinked copolymers of methacrylic acid and of ethyl acrylate.

The crosslinked anionic copolymer may notably be in the form of a dispersion in water. The number-average size of the copolymer particles in the dispersion is generally between 10 and 500 nm, preferably between 20 and 200 nm and more preferentially from 50 to 150 nm.

These copolymers are notably described in patent application WO 01/76552.

More particularly, a crosslinked copolymer of methacrylic acid and ethyl acrylate in the form of a 30% aqueous dispersion manufactured and sold under the name Carbopol Aqua SF-1 by the company Noveon or else a crosslinked copolymer of (meth)acrylic acid and C₁-C₄ alkyl acrylate sold under the name Aculyn 33 by the company Rohm & Haas may be used.

Preferably, the total content of crosslinked anionic copolymer(s) according to the invention ranges from 0.05% to 10% by weight, more preferentially from 0.1% to 5% by weight, even more preferentially from 0.5% to 3% by weight, and better still from 1% to 2% by weight, relative to the total weight of the composition.

Preferably, the total content of copolymers comprising one or more monomers A chosen from acrylic acid and methacrylic acid, one or more monomers of formula b1) as defined above and one or more crosslinking agents ranges from 0.05% to 10% by weight, more preferentially from 0.1% to 5% by weight, even more preferentially from 0.5% to 3% by weight, and better still from 1% to 2% by weight, relative to the total weight of the composition.

Preferably the total content of copolymers chosen from crosslinked copolymers of methacrylic acid and C₁-C₄ alkyl acrylate and crosslinked copolymers of acrylic acid and C₁-C₄ alkyl acrylate, preferably crosslinked copolymers of methacrylic acid and ethyl acrylate, ranges from 0.05% to 10% by weight, more preferentially from 0.1% to 5% by weight, even more preferentially from 0.5% to 3% by weight, and better still from 1% to 2% by weight, relative to the total weight of the composition.

### Nonionic Surfactants

The composition according to the present invention may also comprise one or more nonionic surfactants.

By way of example, the nonionic surfactants may be chosen from:
alcohols, α-diols and (C₁-C₂₀)alkylphenols, these compounds being polyethoxylated and/or polypropoxylated and/or polyglycerolated, the number of ethylene oxide and/or propylene oxide groups possibly ranging from 1 to 100, and the number of glycerol groups possibly ranging from 2 to 30; or else these compounds comprising at least one fatty chain including from 8 to 40 carbon atoms and notably from 16 to 30 carbon atoms; in particular, oxyethylenated saturated or unsaturated, linear or branched alcohols comprising at least one C₈ to C₄₀ alkyl chain, comprising from 1 to 100 mol of ethylene oxide, preferably from 2 to 50 and more particularly from 2 to 40 mol of ethylene oxide and including one or two fatty chains;
condensates of ethylene oxide and propylene oxide with fatty alcohols;
polyethoxylated fatty amides preferably containing from 2 to 30 ethylene oxide units, polyglycerolated fatty amides including on average from 1 to 5 and in particular from 1.5 to 4 glycerol groups;
ethoxylated fatty acid esters of sorbitan, preferably containing from 2 to 40 ethylene oxide units;
fatty acid esters of sucrose;
polyoxyalkylenated, preferably polyoxyethylenated, fatty acid esters containing from 2 to 150 mol of ethylene oxide, including oxyethylenated plant oils;
N—(C₆-C₂₄ alkyl)glucamine derivatives;
amine oxides such as (C₁₀-C₁₄ alkyl)amine oxides or N—(C₁₀-C₁₄ acyl)aminopropylmorpholine oxides;
and mixtures thereof.

When they are present, the total content of the nonionic surfactant(s) ranges from 0.5% to 25% by weight, more preferentially from 1% to 20% by weight, better still from 5% to 15% by weight, relative to the total weight of the composition.

### Cationic Polymers

Preferably, the composition according to the present invention also comprises one or more cationic polymers.

For the purposes of the present invention, the term “cationic polymer” denotes any non-silicone (not comprising any silicon atoms) polymer containing cationic groups and/or groups that can be ionized into cationic groups and not containing any anionic groups and/or groups that can be ionized into anionic groups.

The cationic polymers are not silicone-based (they do not comprise any Si—O units).

The cationic polymers may be associative or non-associative.

Preferably, the cationic polymer(s) are chosen from non-associative cationic polymers.

The cationic polymers that may be used preferably have a weight-average molar mass (Mw) of between 500 and 5×10⁶ approximately and preferably between 10³ and 3×10⁶ approximately.

Among the cationic polymers, mention may be made more particularly of:
(1) homopolymers or copolymers derived from acrylic or methacrylic esters or amides and including at least one of the units having the following formulae:

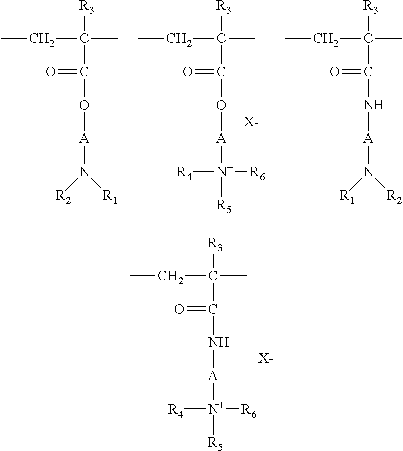

in which formulae:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical, and preferably an alkyl group containing from 1 to 6 carbon atoms;
R₁ and R₂, which may be identical or different, represent a hydrogen atom or an alkyl group containing from 1 to 6 carbon atoms, preferably methyl or ethyl; and
X denotes an anion derived from a mineral or organic acid, such as a methosulfate anion or a halide such as chloride or bromide.

The copolymers of family (1) may also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic acids or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, such as the products sold under the name Bina Quat P 100 by the company Ciba Geigy,
   the copolymer of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in detail in French patents 2 077 143 and 2 393 573,
   dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the products sold under the name Styleze CC 10 by ISP;
   quaternized vinylpyrrolidone/dimethylaminopropylmethacrylamide copolymers such as the product sold under the name Gafquat HS 100 by the company ISP;
   polymers, preferably crosslinked polymers, of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba;
(2) cationic polysaccharides, notably cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as quaternary ammoniums of hydroxyethylcellulose that have reacted with an epoxide substituted with a trimethylammonium group, for instance Polyquaternium-10.

Cationic cellulose copolymers or cellulose derivatives grafted with a water-soluble quaternary ammonium monomer are notably described in patent U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt, for instance Polyquaternium-4. The commercial products corresponding to this definition are more particularly the products sold under the names Celquat L 200 and Celquat H 100 by the company National Starch.

Among the cationic cellulose derivatives, use may also be made of cationic associative celluloses, which may be chosen from quaternized cellulose derivatives, and in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethylcelluloses of formula (Ib):

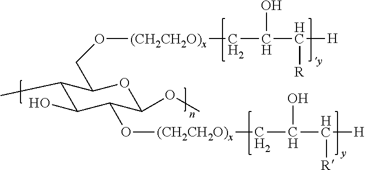

in which:
R represents an ammonium group RaRbRcN⁺—, Q⁻ in which Rₐ, R_{b} and R_{c}, which may be identical or different, represent a hydrogen atom or a linear or branched C₁ to C₃₀ alkyl, preferably an alkyl, and Q⁻ represents an anionic counterion such as a halide, for instance a chloride or bromide;
R′ represents an ammonium group R′aR′bR′cN⁺—, Q′⁻ in which R′a, R′b and R′c, which may be identical or different, represent a hydrogen atom or a linear or branched C₁ to C₃₀ alkyl, preferably an alkyl, and Q′⁻ represents an anionic counterion such as a halide, for instance a chloride or bromide;
it being understood that at least one of the radicals Rₐ, R_{b}, R_{c}, R′a, R′b and R′c represents a linear or branched C₈ to C₃₀ alkyl;
n, x and y, which may be identical or different, represent an integer of between 1 and 10 000.

Preferably, in formula (Ib), at least one of the radicals Rₐ, R_{b}, R_{c}, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radical(s) represent a linear or branched C₁-C₄ alkyl, notably methyl.

Preferably, in formula (Ib), only one of the radicals Rₐ, R_{b}, R_{c}, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radicals represent a linear or branched C₁ to C₄ alkyl, notably methyl.

Better still, R may be a group chosen from —N⁺(CH₃)₃, Q′⁻ and
N⁺(C₁₂H₂₅)(CH₃)₂, Q′⁻, preferably a group —N⁺(CH₃)₃, Q′⁻.

Even better still, R′ may be a group —N⁺(C₁₂H₂₅)(CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200®, sold by the company Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (Cis alkyl), such as the product Crodacel QS®, sold by the company Croda.

Mention may also be made of the hydroxyethylcelluloses of formula (Ib) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 and SL-5, from the company Amerchol/Dow Chemical.

More particularly, the polymers of formula (Ib) are, for example, those the viscosity of which is between 2000 and 3000 cPs inclusive, preferentially between 2700 and 2800 cPs. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs. Use may also be made of Softcat Polymer SX-1300X with a viscosity of between 1000 and 2000 cPs.

The cationic galactomannan gums are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are notably sold under the names Jaguar C13 S, Jaguar C 15, Jaguar C 17, Jaguar C162 or Jaguar Excel by the company Rhodia. Such compounds have the INCI name guar hydroxypropyltrimonium chloride or hydroxypropyl guar hydroxypropyltrimonium chloride.
(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.
(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they include one or more tertiary amine functions, they can be quaternized.
(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.
(6) polymers obtained by reacting a polyalkylene polyamine including two primary amine groups and at least one secondary amine group with a dicarboxylic acid chosen from diglycolic acid and saturated aliphatic dicarboxylic acids having from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or under the name PD 170 or Delsette 101 by the company Hercules in the case of the adipic acid/epoxy-propyl/diethylenetriamine copolymer.
(7) cyclopolymers of alkyldiallylamine or of dialkyldiallylammonium, such as homopolymers or copolymers including, as main constituent of the chain, units corresponding to formula (VI) or (VII):

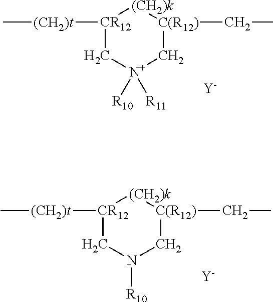

in which formulae (VI) and (VII):
k and t are equal to 0 or 1, the sum k+t being equal to 1;
R₁₂ denotes a hydrogen atom or a methyl radical;
R₁₀ and R₁₁, independently of one another, denote an alkyl group containing from 1 to 6 carbon atoms, a hydroxyalkyl group in which the alkyl group contains 1 to 5 carbon atoms, a C₁ to C₄ amidoalkyl group; or alternatively R₁₀ and R₁₁ may denote, together with the nitrogen atom to which they are attached, heterocyclic groups such as piperidinyl or morpholinyl; R₁₀ and R₁₁, independently of one another, preferably denote an alkyl group containing from 1 to 4 carbon atoms; and
Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer sold, for example, under the name Merquat 100 by the company Nalco (and homologues thereof of low weight-average molar masses) and the copolymers of diallyldimethylammonium salts (for example chloride) and of acrylamide, notably sold under the names Merquat 550 and Merquat 7SPR;
(8) quaternary diammonium polymers comprising repeating units of formula:

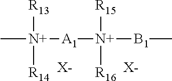

in which formula (VIII):
R₁₃, R₁₄, R₁₅ and R₁₆, which may be identical or different, represent aliphatic, alicyclic or arylaliphatic radicals comprising from 1 to 20 carbon atoms or lower (preferably C₁-C₆) hydroxyalkylaliphatic radicals, or alternatively R₁₃, R₁₄, R₁₅ and R₁₆, together or separately, constitute, with the nitrogen atoms to which they are attached, heterocycles optionally comprising a second heteroatom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇.D where R₁₇ is an alkylene and D is a quaternary ammonium group;
A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a linear or branched, saturated or unsaturated alkylene or hydroxyalkylene radical, B1 can also denote a group (CH₂)ₙCO-D-OC—(CH₂)ₙ— in which D denotes:
a) a glycol residue of formula —O—Z—O—, in which Z denotes a linear or branched hydrocarbon-based radical or a group corresponding to one of the following formulae: —(CH₂—CH₂—O)ₓ—CH₂—CH₂— and —[CH₂CH(CH₃)—O]_{y}—CH₂—CH(CH₃)—, where x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula: —NH—Y—NH—, where Y denotes a linear or branched hydrocarbon-based radical, or alternatively the divalent radical —CH₂—CH₂—S—S—CH₂—CH₂—; or
d) a ureylene group of formula: —NH—CO—NH—.

Preferably, X⁻ is an anion such as chloride or bromide. These polymers have a number-average molar mass (Mn) generally of between 1000 and 100 000.

Mention may be made more particularly of polymers which are constituted of repeating units corresponding to the formula:

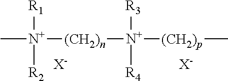

in which formula (IX) R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (IX) that is particularly preferred is the one for which R₁, R₂, R₃ and R₄ represent a methyl radical and n=3, p=6 and X=Cl, which is known as Hexadimethrine chloride according to the INCI (CTFA) nomenclature;
(9) polyquaternary ammonium polymers comprising units of formula (X):

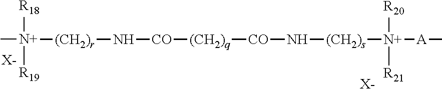

in which formula (X):
R₁₈, R₁₉, R₂₀ and R₂₁, which may be identical or different, represent a hydrogen atom or a methyl, ethyl, propyl, O-hydroxyethyl, (3-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion, such as a halide, and
A denotes a dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by the company Miranol;
(10) quaternary polymers of vinylpyrrolidone and of vinylimidazole, for instance the products sold under the names Luviquat® FC 905, FC 550 and FC 370 by the company BASF;
(11) polyamines such as Polyquart® H sold by Cognis, which is referenced under the name Polyethylene Glycol (15) Tallow Polyamine in the CTFA dictionary;
(12) polymers including in their structure:
(a) one or more units corresponding to formula (A) below:

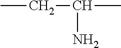

(b) optionally one or more units corresponding to formula (B) below:

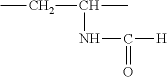

In other words, these polymers may be particularly chosen from homopolymers or copolymers including one or more units derived from vinylamine and optionally one or more units derived from vinylformamide.

Preferably, these cationic polymers are chosen from polymers including, in their structure, from 5 mol % to 100 mol % of units corresponding to formula (A) and from 0 to 95 mol % of units corresponding to formula (B), preferentially from 10 mol % to 100 mol % of units corresponding to formula (A) and from 0 to 90 mol % of units corresponding to formula (B).

These polymers may be obtained, for example, by partial hydrolysis of polyvinylformamide. This hydrolysis may take place in acidic or basic medium.

The weight-average molecular mass of said polymer, measured by light scattering, may range preferably from 1000 to 3 000 000 g/mol, more preferentially from 10 000 to 1 000 000 and even more particularly from 100 000 to 500 000 g/mol.

The cationic charge density of these polymers may preferably range from 2 meq/g to 20 meq/g, more preferentially from 2.5 to 15 meq/g and more particularly from 3.5 to 10 meq/g.

The polymers including units of formula (A) and optionally units of formula (B) are particularly sold under the name Lupamin by the company BASF; for instance, in a non-limiting manner, the products sold under the names Lupamin 9095, Lupamin 5095, Lupamin 1095, Lupamin 9030 (or Luviquat 9030) and Lupamin 9010.
(13) and mixtures thereof.

Preferably, the cationic polymer(s) are chosen from cationic polysaccharides, in particular associative or non-associative cationic polysaccharides; more preferentially from non-associative cationic polysaccharides.

More preferentially, the cationic polymer(s) are chosen from cellulose ether derivatives including quaternary ammonium groups, cationic cellulose copolymers, cellulose derivatives grafted with a water-soluble quaternary ammonium monomer and cationic galactomannan gums, and mixtures thereof.

Even more preferentially, the cationic polymer(s) are chosen from cellulose ether derivatives containing quaternary ammonium groups, and even more preferentially the composition according to the invention contains polyquaternium-10.

Preferably, when they are present in the composition, the total content of cationic polymer(s) ranges from 0.01% to 10% by weight, more preferentially from 0.05% to 5% by weight, even more preferentially from 0.1% to 3% by weight, better still from 0.1% to 1% by weight, relative to the total weight of the composition.

Preferably, when they are present in the composition, the total content of cationic polysaccharide(s) ranges from 0.01% to 10% by weight, more preferentially from 0.05% to 5% by weight, even more preferentially from 0.1% to 3% by weight, better still from 0.1% to 1% by weight, relative to the total weight of the composition.

Preferably, when they are present in the composition, the total content of cellulose ether derivatives including quaternary ammonium groups, preferably polyquaternium-10, ranges from 0.01% to 10% by weight, more preferentially from 0.05% to 5% by weight, even more preferentially from 0.1% to 3% by weight, better still from 0.2% to 1% by weight, relative to the total weight of the composition.

Preferably, the composition according to the invention also comprises at least one inorganic salt, more preferentially sodium chloride.

For the purposes of the present application, the inorganic salt(s) are different from the anionic surfactants (i) and from the amphoteric or zwitterionic surfactants (ii), from the particles, and more particularly from all the ingredients described above.

Preferably, the total content of inorganic salt(s) present in the composition according to the invention, ranges from 0.01% to 5% by weight, more preferentially from 0.05% to 3% by weight and even more preferentially from 0.1% to 2% by weight relative to the total weight of the composition.

Preferably, the composition according to the invention comprises water, that is to say that the medium of the composition is aqueous or aqueous-alcoholic.

More preferentially, the total content of water in the composition according to the invention is between 20% and 98% by weight, more preferentially between 50% and 96% by weight, even more preferentially between 60% and 95% by weight, better still between 65% and 90% by weight, relative to the total weight of the composition.

The composition according to the invention may also comprise at least one organic solvent.

For the purposes of the invention, it is understood that the organic solvents are liquid at 25 °C and at atmospheric pressure.

By way of examples of organic solvent, use may particularly be made of those which are water-soluble, such as C₁-C₇ alcohols, and in particular C₁-C₇ aliphatic or aromatic mono alcohols, C₃-C₇ polyols and C₃-C₇ polyol ethers, which can be used alone or as a mixture with water. Advantageously, the organic solvent(s) may be chosen from ethanol, isopropanol, propylene glycol, hexylene glycol and glycerol, and mixtures thereof.

Preferably, the composition according to the invention comprises at least one C₂-C₇ and more preferentially C₂-C₆ polyol, even more preferentially glycerol.

Preferably, when the organic solvent(s) are present in the composition according to the invention, the total content of C₂-C₇ organic solvent(s) is between 0.5% and 10% by weight, more preferentially between 1% and 5% by weight, relative to the total weight of the composition.

Preferably, when the composition according to the invention comprises water, the pH of the composition is between 3.0 and 9.0, more preferentially between 3.5 and 8.0, even more preferentially between 4.0 and 7.0, better still between 4.5 and 6.5.

The pH of the composition according to the invention may be adjusted to the desired value by means of basifying agents (such as monoethanolamine) and/or acidifying agents (such as citric acid).

The composition according to the invention may also optionally contain one or more additives used in cosmetics, such as fragrances, thickeners other than the polymers described previously, and dyes.

These additives may be present in the composition according to the invention in an amount ranging from 0 to 20% by weight, relative to the total weight of the composition.

A person skilled in the art will take care to select these optional additives and amounts thereof so that they do not harm the properties of the compositions of the present invention.

The composition according to the invention may advantageously be in the form of a shampoo.

The viscosity of the composition according to the invention may be determined using a viscometer such as a Rheomat RM100 device from the company Lamy Rheology (measurement at 25 °C, 30 seconds, rotation speed 200 rpm, spindle 4).

Preferably, the viscosity of the composition ranges from 2000 to 10 000 mPa·s, preferentially from 4000 to 9500 mPa·s, better still from 5000 to 9000 mPa·s, better still from 5500 to 8500 mPa·s, even better still from 6000 to 8300 mPa·s.

The invention also relates to a process for treating, notably cosmetically, keratin materials, in particular human keratin materials such as the hair and the scalp, comprising at least one step of applying a composition as defined previously to said materials.

Preferably, the process according to the invention is a process for washing and/or conditioning human keratin materials, more particularly a process for washing the hair and the scalp, comprising at least one step of applying to the keratin materials a composition as defined previously, followed by a step of rubbing said materials by means of the composition.

According to a preferred embodiment of the invention, a step of rinsing the keratin fibres is then performed.

A step of drying the keratin fibres may also be envisaged in the process according to the invention, notably using a heating means such as a hair dryer, a straightening iron, a steam iron or a heating hood, the heating means possibly heating to a temperature ranging from 35 °C to 230 °C, preferably from 50 to 120 °C.

Finally, a subject of the invention is the use of the composition as described previously for treating keratin materials, preferably for washing the hair and the scalp.

The example that follows serves to illustrate the invention without, however, being limiting in nature.

### EXAMPLE

Compositions A and B were prepared from the ingredients indicated in the table below, the amounts of which are expressed as weight percentages of active material (AM).

TABLE 1

| Ingredients | A | B |
|---|---|---|
| Sodium laureth sulfate | 10.8 | 10.8 |
| Sodium lauryl sulfate | 3.4 | 3.4 |
| Salicylic acid | 1.8 | 1.8 |
| Cocoyl betaine | 1.8 | 1.8 |
| Laureth-5 carboxylic acid | 1.8 | 1.8 |
| Cocamide MEA | 2 | 2 |
| Crosslinked copolymer of methacrylic | 1.5 | — |
| acid and ethyl acrylate (1) | | |
| Crosslinked acrylic acid homopolymer (2) | — | 1.5 |
| Cocamide MIPA | 1.0 | 1.0 |
| Apricot kernel powder (3) | 0.5 | 0.5 |
| Argan nut shell powder (4) | 0.5 | 0.5 |
| Sodium chloride | 0.4 | 0.4 |
| Polyquaternium-10 | 0.3 | 0.3 |
| Isopropyl myristate | 0.3 | 0.3 |
| Polyols | 2.8 | 2.8 |
| PEG-55 propylene glycol oleate | 0.2 | 0.2 |
| Preserving agents, pH agents, fragrance | qs | qs |
| Water | qs 100 | qs 100 |

(1) INCI name: Acrylates Copolymer, Carbopol Aqua SF-1 sold by the company Noveon
(2) INCI name: Carbomer, Carbopol 980 sold by the company Lubrizol
(3) INCI name: Prunus armeniaca Seed Powder (number-average diameter of between 300 and 400 μm)
(4) INCI name: Argania spinosa Shell Powder (number-average diameter of between 410 and 800 μm).

Composition A is in accordance with the invention, composition B is comparative.

The two compositions were prepared by homogeneous mixing of the ingredients at room temperature (25 °C). The stability of the two compositions was then assessed by placing them for two months in an oven maintained at a temperature of 45 °C and in the dark.

After one month of storage, the appearance of composition A was unchanged, and it was in the form of a visually homogeneous suspension of solid particles in a clear fluid. Composition B, on the other hand, showed the beginnings of phase separation: there were no more particles on the surface of the composition, an upper layer of particle-free composition had formed and the fluid was cloudy.

After two months of storage at 45 °C, the appearance of composition A still remained unchanged: it was in the form of a visually homogeneous suspension of solid particles in a clear fluid and no phase separation was visible. In the case of composition B, the phase separation increased, the thickness of the upper layer of particle-free composition increased and the fluid was cloudy.

It is therefore seen that composition A according to the present invention has better stability over time than the comparative composition B.

1. Cosmetic composition comprising:
(i) salicylic acid and/or at least one salicylic acid salt, in a total content of at least 1.5% by weight relative to the total weight of the composition,
(ii) solid particles with a number-average size of greater than or equal to 300 μm, in a total content of at least 0.2% by weight relative to the total weight of the composition,
(iii) at least one anionic surfactant,
(iv) at least one amphoteric or zwitterionic surfactant, and
(v) at least one crosslinked anionic copolymer of at least one monomer A chosen from vinyl carboxylic acids and of at least one monomer B containing at least one α,β-ethylenic unsaturation chosen from monomers of the following formulae:
CH₂═CXY b1)
where X denotes hydrogen and Y denotes a group chosen from —COOR, —C₆H₄R′, —CN, —CONH₂, —NHCOCH₃, —CONHC(CH₃)₃, and —CON(CH₃)₂, or
X denotes CH₃ and Y denotes a group chosen from —COOR, —C₆H₄R′, —CN or —CH═CH₂,
R denoting a C₅-C₈ alkyl group or a C₂-C₈ hydroxyalkyl group, preferably a C₁-C₄ alkyl group or a C₂-C₄ hydroxyalkyl group,
R′ denoting hydrogen, a C₁-C₈ alkyl group or a C₂-C₈ hydroxyalkyl group;
CH₂═CH(OCOR¹) b2)
with R¹ denoting a C₁-C₈ and preferably C₁-C₄ alkyl group;
CH₂═CH₂ or CH₂═CHCH₃. b3)

2. Composition according to claim 1, characterized in that it comprises salicylic acid and/or at least one salicylic acid salt in a total content ranging from 1.5% to 8% by weight, preferably from 1.8% to 6% by weight and better still from 2% to 5% by weight relative to the total weight of the composition.

3. Composition according to claim 1, characterized in that the number-average size of the particles is in the range from 300 to 1500 μm, preferably from 350 to 1000 μm and better still from 400 to 800 μm.

4. Composition according to claim 1, characterized in that the solid particles are chosen from organic particles and more preferentially from fragments of one or more plants.

5. Composition according to claim 1, characterized in that it comprises at least one plant material powder, preferably chosen from kernel powders such as apricot kernel powder, plum stone powder, olive stone powder; nut shell powders such as argan nut shell powder (INCI name: Argania spinosa shell powder), nut shell powder; seed powders such as coffee bean powder, cocoa bean powder; seed powders such as kiwi seed powder; and also mixtures of these powders; preferably the composition comprises a kernel powder and a nut shell powder; better still an apricot kernel powder and an argan nut shell powder.

6. Composition according to claim 1, characterized in that it comprises said solid particles in a total content ranging from 0.2% to 10% by weight, preferably from 0.5% to 5% by weight, better still from 0.7% to 2% by weight and even better still from 0.8% to 1.5% by weight relative to the total weight of the composition.

7. Composition according to claim 1, characterized in that it comprises at least one carboxylate anionic surfactant, preferably chosen from: acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and mixtures thereof;
the alkyl and/or acyl groups of these compounds including from 6 to 30 carbon atoms, notably from 12 to 28, even better still from 14 to 24 or even from 16 to 22 carbon atoms; the aryl group preferably denoting a phenyl or benzyl group;
these compounds possibly being polyoxyalkylenated, notably polyoxyethylenated, and then preferably including from 1 to 50 ethylene oxide units and better still from 2 to 10 ethylene oxide units;
and more preferentially chosen from (C₆-C₂₄)alkyl ether carboxylic acids, and better still (C₁₂-C₂₀)alkyl ether carboxylic acids.

8. Composition according to claim 1, characterized in that it comprises at least one sulfate anionic surfactant, preferably chosen from alkyl sulfates, alkyl ether sulfates, alkyl amido ether sulfates, alkyl aryl polyether sulfates, monoglyceride sulfates, the salts of these compounds, and mixtures thereof;
the alkyl groups of these compounds preferably comprising from 6 to 30 carbon atoms, notably from 8 to 28, even better still from 10 to 24 or even from 12 to 22 carbon atoms; the aryl group preferably denoting a phenyl or benzyl group;
these compounds possibly being (poly)oxyalkylenated, notably (poly)oxyethylenated, and then preferably including from 1 to 50 ethylene oxide units and better still from 1 to 10 ethylene oxide units;
and more preferentially chosen from:
C₆-C₃₀, better still C₈-C₂₄ and even better still C₁₀-C₂₄ alkyl sulfates,
C₆-C₂₄ and better still C₁₀-C₂₄ alkyl ether sulfates; preferably comprising from 1 to 20 ethylene oxide units;
the salts of these compounds and mixtures thereof.

9. Composition according to claim 1, characterized in that the total content of anionic surfactant(s) ranges from 1% to 25% by weight, preferably from 2% to 20% by weight, more preferentially from 5% to 18% by weight and better still from 10% to 18% by weight relative to the total weight of the composition.

10. Composition according to claim 1, characterized in that the amphoteric or zwitterionic surfactants are chosen from (C₈-C₂₀)alkylbetaines, (C₈-C₂₀)alkylamido(C₃-C₈)alkylbetaines, (C₈-C₂₀)alkylamphoacetates, (C₈-C₂₀)alkylamphodiacetates and mixtures thereof, preferably from (C₈-C₂₀)alkylbetaines, (C₈-C₂₀)alkylamido(C₃-C₈)alkylbetaines and mixtures thereof, even more preferentially from cocoyl betaine, cocamidopropyl betaine and mixtures thereof.

11. Composition according to claim 1, characterized in that the total content of amphoteric or zwitterionic surfactant(s) ranges from 0.1% to 15% by weight, preferably from 0.5% to 10% by weight and more preferentially from 1% to 5% by weight relative to the total weight of the composition.

12. Composition according to claim 1, characterized in that the crosslinked anionic copolymer is derived from at least one monomer A chosen from acrylic acid, methacrylic acid, itaconic acid, fumaric acid, crotonic acid, maleic acid and mixtures thereof; and from at least one monomer B of formula b1).

13. Composition according to claim 1, characterized in that the crosslinked anionic copolymer is chosen from crosslinked copolymers of methacrylic acid and C₁-C₄ alkyl acrylate, crosslinked copolymers of acrylic acid and C₁-C₄ alkyl acrylate, and preferably crosslinked copolymers of methacrylic acid and ethyl acrylate.

14. Composition according to claim 1, characterized in that the total content of crosslinked anionic copolymer(s) ranges from 0.05% to 10% by weight, preferably from 0.1% to 5% by weight, more preferentially from 0.5% to 3% by weight and better still from 1% to 2% by weight relative to the total weight of the composition.

15. Process for treating keratin materials, preferably for washing and/or conditioning keratin materials, comprising at least one step of applying the composition as defined in claim 1 to said materials.
